(19) United States
(12) Patent Application Publication
(10) Pub. No.: US 2005/0003518 A1
(43) Pub. Date: Jan. 6, 2005

(54) Adapter for multi-element contact-probe

(76) Inventors: David Aviram, Tel-Aviv (IL); Zohar Avrahami, Tel-Aviv (IL)

Correspondence Address: MARTIN MOYNIHAN;c/o ANTHONY CASTORINA, SUITE 207, 2001 JEFFERSON DAVIS HIGHWAY, ARLINGTON, VA, 22202, US

(21) Appl. No.: 10/863,746

(22) Filed: Jun. 9, 2004

(30) Foreign Application Priority Data
Jul. 6, 2003 (IL) 156789

(51) Int. Cl. C12M 1/34

(52) U.S. Cl. 435/287.1

(57) ABSTRACT

The disclosed invention describes an Adaptor structure, used in conjunction with a multi-element contact-probe, for protecting the probe elements and the Target surface from wearing and contamination, while maintaining probe functionality including the probe elements contact force exerted on the corresponding Target surface points. The provided Adaptor structure is removable and suitable for disposable applications. The invention constitutes also a method in which the mediating Adaptor structure physically buffers between the Contact probe front-end and Target surface. Further the invention constitutes also a kit comprising at least one Contact probe and at least one Adaptor structure.

This application claims the benefit of priority from IL Patent application No. 156789, filed Jul. 6, 2003.

### FIELD OF THE INVENTION

This present invention relates to disposable adaptors for intermediating between multi elements contact-probe and a Target surface, involving transfer of energy, Substance or force across the adaptor thickness to or from the Target surface, for protecting the probe or the Target surface, both or either one.

### PRIOR ART OF RELEVANCE

The following U.S. Pat. No.: 6,489,003 to 3M; U.S. Pat. No. 6,508,785 to Altea; and U.S. Pat. Nos. 6,148,232 and 5,983,135 both to Transpharma include prior art of relevance.

### BACKGROUND OF THE INVENTION

Contact array probes are widely used in industry and medical applications. In the electronics industry Contact probes are used in variety of testing systems usually by utilizing fixtures called “bed of nails” to provide electrical contacts with test pads on boards. Likewise probes of bioelectric mapping are used in medical applications to provide electrical contact with skin or tissue. Similarly probes used to ablate tissue at plurality of points for drug delivery or analyte extraction applications are known (for example Altea Technologies transdermal device which transfers heat to the skin U.S. Pat. No. 6,508,785, Transpharma Medical Viaderm which transfers RF energy to the skin U.S. Pat. No. 6,148,232). Ultrasound probes, microphone Arrays for auscultation, multi-element vibrators, spatial temperature probes, drug delivery systems, etc. all require physical contact with the Target surface at all points of interaction.

As known to any professional in the field, the lack of contact force adversely affects the energy or Substance transfer to and from the active element and the Target surface. An Array structure that ensures determined contact force of each of the Array elements is quite complex and expensive. Upon exerting force at a given point on an object surface a local deformation is introduced ranging spatially in the neighborhood of the contacted point. An adjacent element will not come into contact unless it is able to travel in order to form a physical contact with the object. It is therefore required to utilize a fixture with an independent force actuator along a predetermined displacement range, for each one of the probe elements. As an example “bed of nails” fixture consists plurality of spring loaded pin contacts to allow each contact to engage at different position while exerting the required force, resulting in an expensive fixture prohibiting disposable use. Another example is the medical devices such as ultrasound probes, bioimpedance probes, transdermal skin treatment devices which require a very high-pressure to create such a high internal object stress (pressure) causing deformation of Target surface to ensure each element contact force, resulting in low patient compliance.

When probe front-end surface comes into contact with the Target surface it wears and gets contaminated by the Target surface, requiring maintenance, cleaning and in some applications sterilization, which are costly procedures. In some medical applications it is prohibitive to reuse the probe.

Therefore there is a clear need for a method in which a mediating low cost member is utilized for buffering between the Contact probe and the Target surface; and for a system comprising at least one Contact probe and at least one compatible mediating low cost disposable adaptor which introduces a protective buffer between the probe and Target surface, as well as, for an replaceable adaptor suitable for maintaining probe functionality.

### SUMMARY OF THE INVENTION

Definitions:

For the purpose of this invention the following definitions in addition to ordinary context, shall apply:

A Contact probe shall mean a device for delivering or receiving energy or Substance, consisting of multi elements front end, that for proper operation requires physical contact with a Target surface (defined below), with a contact force greater than a predetermined value.

A Target surface shall mean the contacted, monitored or treated, object surface, which may be either continuous or a surface with discrete contact zones. For non-limiting examples, electronic printed circuits or IC wafers with discrete test pads, human skin, biological tissues or industrial layers.

An Array shall mean plurality (at least two) of elements organized in line. or planar or 3D surface arrangement.

An Adaptor structure shall mean a self-supporting structure adapted to be mounted on a said Contact probe or a said Target surface, consisting of a frame structure, which supports a sheet-structure. The Adaptor structure when used in alignment with a Contact probe will form a physical buffer between the Contact probe front-end elements and the Target surface while substantially maintaining the functionality of the said Contact probe.

An Element of said multi element front end shall mean a functional element Contact probe, for example electrode, piezoelectric element, LED, lenses, heating element, etc.

A Sheet-structure shall mean a deformable structure characterized by its dimensional aspect ratio where the thickness is significantly smaller than the other dimensions. A Sheet structure consists of plurality of Elastically coupled (defined below) More rigid constituents (defined below). The Sheet structure is further characterized by its ability to transfer localized contact force, exerted by a Contact probe Element, to the corresponding point on the Target surface, as well as, by providing a functional path between a Contact probe front-end Element and the corresponding Target surface point.

A More rigid constituent shall mean a zone within the said Sheet structure characterized by enabling local transfer of mechanical force across the sheet-structure. A More rigid constituent may include additional functional members (as further described) such as: electrical conducting members, heat conducting members, Substance permeable members and electromagnetic radiation transmission members.

An Elastically coupling shall mean joining means or material surrounding the More rigid constituents, allowing locally confined deformation of said Sheet structure (preferably elastic) substantially in the normal orientation by enabling displacement of the corresponding More rigid constituents upon exertion of a local force. Elastically coupling may be implemented at various methods, as non limiting examples: introducing non-through fractures around More rigid constituents, reduced material thickness in between More rigid constituents, use of highly elastic or highly flexible materials and by controlled polymerization of joining zones.

A Reservoir shall mean a pocket within the said More rigid constituents, that may consist controlled opening and is adapted to store Substance in the form of—liquid, solid, or powder.

Substance shall mean material to be delivered or extracted to or from the Target surface object. As non-limiting examples: drug, paint, inter cellular liquid etc.

Kit shall mean a system comprising of at least one Contact probe and at least one Adaptor structure.

The present invention provides a sheet structure wherein the More rigid constituents are in alignment with a Contact probe front end Elements to transfer the force exerted by each probe front end Element to the corresponding point on the Target surface. The More rigid constituents are joined together to form a Sheet structure. The said Sheet structure is adapted to provide a physical buffer between the probe front end Elements and Target surface, preferably simple enough for disposable use. It further suggests, that said More rigid constituents are joined together to form a Sheet structure by Elastically coupling that allow each of the More rigid constituents to be displaced in correspondence with its adjacent elements at a substantially normal orientation to the Sheet structure, while minimizing transversal stress coupling among the More rigid constituent elements, thus significantly reducing the influence on neighboring elements contact force. Apart of transferring force the More rigid constituents are adapted to transfer the appropriate energy form or Substance to allow probe functionality.

One objective of the present invention is to provide a disposable structure adapted for buffering between a Contact probe and a Target surface while maintaining functionality of the Contact probe.

Further objectives of the present invention are to provide a removable Contact probe Adaptor that may be designed in accordance with the specific application to:

1. Enable transfer of substantially each probe Element contact force to or from the spatially corresponding Target surface point.

2. Enable electrical conduction between at least some of probe Elements to or from the spatially corresponding Target surface point.

3. Enable heat conduction between at least some of probe Elements to or from the spatially corresponding Target surface point.

4. Enable transfer of electromagnetic energy between at least some of probe Elements to or from the spatially corresponding Target surface point.

5. Enable transfer of acoustic energy between at least some of probe Elements to or from the spatially corresponding Target surface point.

6. Enable transfer of mechanical vibration energy between at least some of probe Elements to or from the spatially corresponding Target surface point.

7. Enable transfer of Substance between at least some of probe Elements to or from the spatially corresponding Target surface point.

8. Enable delivery of Substance from embedded Reservoir related to at least to some of probe Elements to the spatially corresponding Target surface point.

9. Enable accumulation of Substance into embedded Reservoir related to at least to some of probe Elements, extracted from the spatially corresponding Target surface point.

The present invention provides also a method for buffering between a Contact probe and a surface Target by utilizing a removable mediating member in alignment with Contact probe Elements enabling transfer of contact force while maintaining probe functionality for protecting either the probe or the Target surface from contamination or wear.

The present invention provides also a system, in a form of a kit, composed of at least one Contact probe and at least one Adaptor structure wherein the Adaptor structures may be of different functionality types.

More objectives of the present invention will become apparent from the following description.

The invention is described by no limiting examples and it is appreciated that variations in terms of geometry, materials, configuration, dimensions and functional combinations based on the presented invention are possible and should be considered to be within its scope.

### BRIEF DESCRIPTION OF DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

All of the figures are partial views or cross-sections of the inventive Sheet structure showing various embodiments of the elements and their combinations, as follows:

FIG. 1: General orientation of Contact probe, Adaptor structure and Target surface.

FIG. 2: Example of Sheet structure adhered to Target surface.

FIG. 3: Illustration of fixed Array contact force.

FIG. 4: Illustration of flexible Array contact force.

FIG. 5: Illustration of Adaptor structure principle of operation.

FIG. 6: Illustration of the More rigid constituents construction.

FIG. 7: Illustration of sheet structures.

FIG. 8: Block illustration of the method.

FIG. 9: Block illustration of a Kit.

### DESCRIPTION OF THE PREFERRED EMBODIMENTS

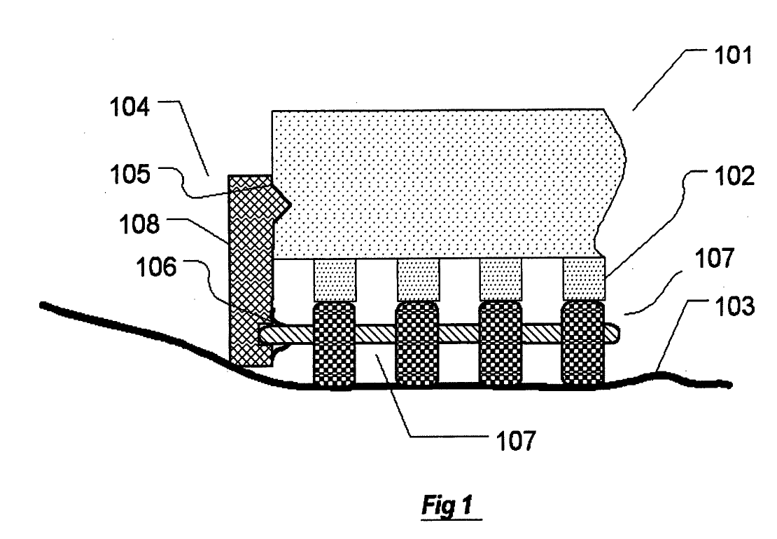

Reference is made to FIG. 1 showing a partial view of a Contact probe, Adaptor structure and Target surface. A Contact probe 101 consisting of at least some active Elements 102 attached to a Target surface 103 through an Adaptor structure 104. The Adaptor structure 104 is adapted to be mounted on the Contact probe 101 in alignment with its active Elements by fastening means 105 which may be implemented in various ways such as mechanical clamps, mechanical fasteners, magnetic fastener, pin guiders and locking mechanisms known to any professional person in the field. The Adaptor structure 104 basically consists of a frame structure 108 made of a rigid material, preferably injected plastic material compatible with the specific application, to provide a support structure to which a Sheet structure 101 is attached by common means 108, such as: ultrasound welding, adhesion, mechanical fastening or being molded as an integral part of the frame, known to any professional person in the field.

It is appreciated that one required important feature of a Contact probe, among others, for a proper operation is to ensure physical force contact of Elements 102 with the spatially corresponding points on the Target surface, transferred through the Sheet structure 107.

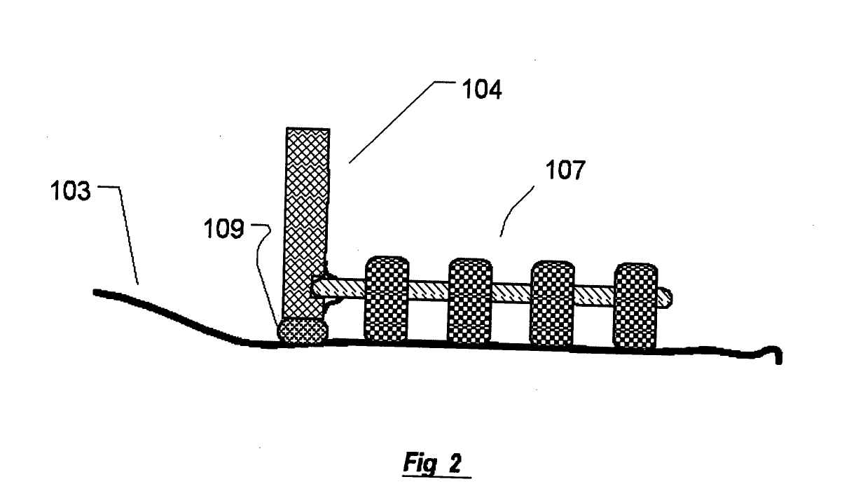

In Fig.2 an alternative configuration is illustrated, wherein the Adaptor structure 104 is adhered by contact adhesion means 109 to the Target surface 103, while upon operation the Contact probe 101 is brought into contact with the Adaptor structure guided by the Adaptor frame structure.

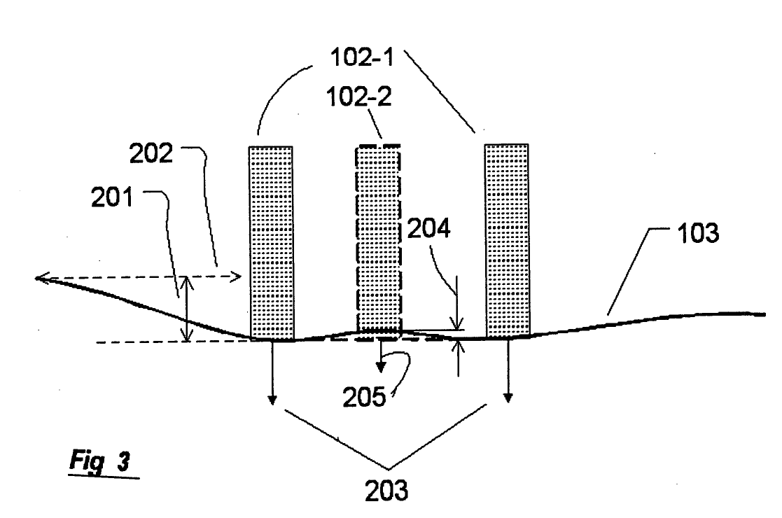

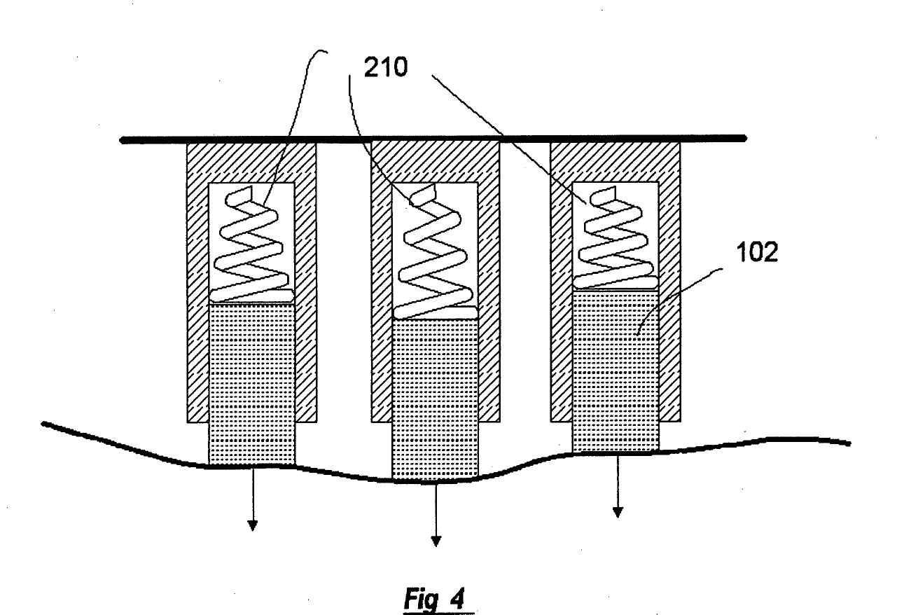

It is appreciated that many target objects are not absolutely rigid and upon exerting force at a point on their surface a local deformation, either plastic or elastic is introduced in the surrounding region. Reference is made to FIG. 3 in which this phenomenon is illustrated. Two contact Elements 102-1 exert a contact force F1 203 on the Target surface 103 causing a local deformation of depth X1 201 extending laterally in the local region denoted by R1 202. This phenomenon is easily observed by applying a point pressure to a human skin. Further notice is made that if an identical additional contact Element 102-2 is located in between the said two Elements, it will only slightly modify the deformation depth denoted by X2 204 while exerting a contact force F2 205 weaker than F1 203. This phenomenon of non-uniform contact force distribution over Array Elements is substantially reduced by individually force-actuated Elements as illustrated in FIG. 4. A force actuator means is attached to a contact Element exerting a determined force over a predetermined displacement range. As an example one may consider a “bed of nails” test fixture used for multi electrical contacts applications wherein the contact Elements are spring loaded 210 acting on movable pin contacts 102. It is therefore appreciated that a probe structure with moveable active Elements, individually force actuated, is relatively more complex and by definition more expensive and in some cases prohibitive for disposable use. It is also appreciated that physical contact is a source of probe Elements wear as well as contamination of Elements and Target surface, both or either one. In order to avoid said drawbacks an intermediate protective Sheet structure 107 is suggested.

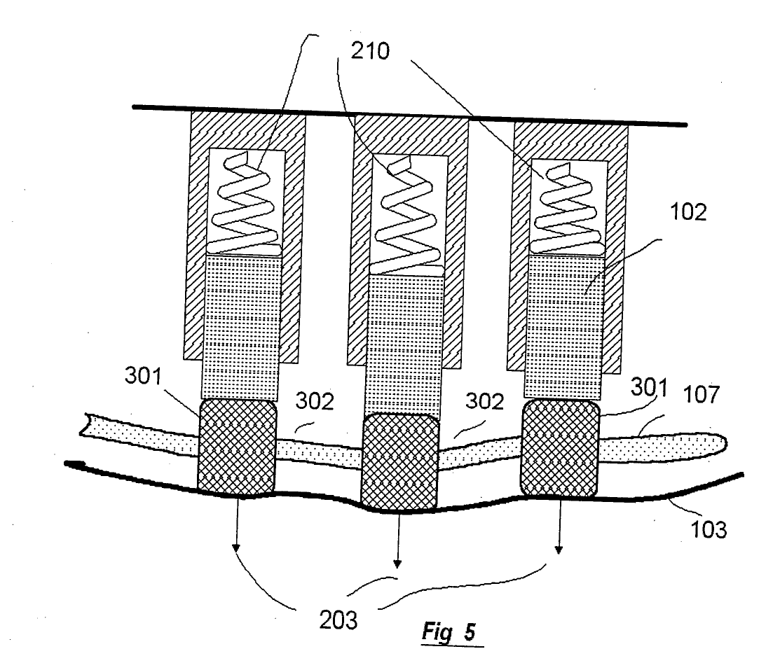

Reference is made to FIG. 5; wherein a partial illustration of probe Elements is shown. Elements 102 engage through a Sheet structure 107 with the Target surface 103. As can be seen from the illustration the Sheet structure 107 is composed of More rigid constituents 301 joined together by Elastically coupling 302 to form the said Sheet structure 107. The Sheet structure construction is described and elaborated later on, at this stage, notice is made to the alignment of the More rigid constituents 301 with the probe Elements 102 which enable transfer of contact force while allowing each More rigid constituent substantially independent displacement in a substantially normal to Sheet structure 107 orientation, thus allowing contact with the corresponding point of the Target surface while exerting a predetermined force 203.

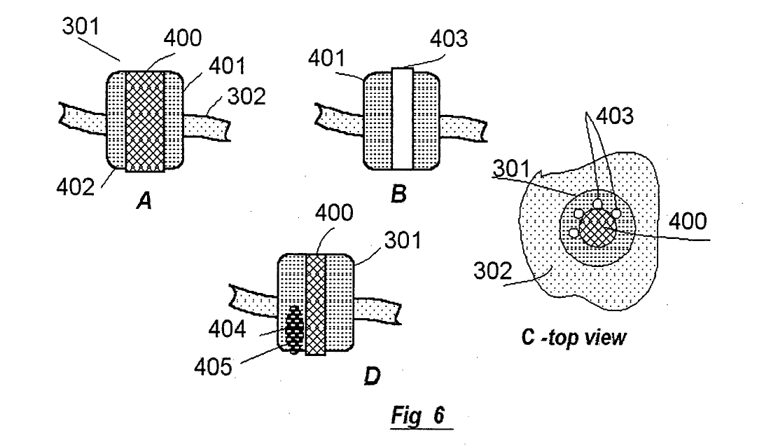

It is further suggested that the More rigid constituents may be designed to include additional functional features as illustrated by way of examples in FIG. 6.

In FIG. 6A a generalized illustration of the More rigid constituent 301 is given. The More rigid constituent 301 comprises of supporting material 401, made of compatible material such as rubber, Silicon rubber, compatible plastic or polymeric material, connected and supported by the Elastically coupling 302 further made of, preferably in terms of simplicity, the same material, but may also be made of different materials more compatible to the application in terms of their mechanical features. It is appreciated that the More rigid constituents 301 are supported by the Elastically coupling member 302, thus enabling substantially independent displacement of each More rigid constituent 301 related to its neighbors within a predetermined range. Further, in the More rigid constituent 301 a functional member 400 may be embedded. Functional member 400, for example may be: electrical conducting material to provide electrical path across the More rigid constituent, heat conducting material to provide heat transfer path across the More rigid constituent, acoustic conducting material to provide acoustic path across the More rigid constituent, wave guide member to provide electromagnetic radiation path across the More rigid constituent and fiber optics element to provide light propagation path across the More rigid constituent. The More rigid constituent 301 may be also coated with adhesion means 402 to adhere to the Target surface.

Further in FIG. 6B a functional member of the type of Substance transfer is illustrated. Member 403 comprises a Substance passage in the form of hollow tube, purposive material or transparent or semi-transparent membrane to allow Substance transfer across the More rigid constituent 301.

FIG. 6C illustrates an example of combination of functional members.

FIG. 6D demonstrates an additional attribute in the form of Substance Reservoir. Pocket 404 may contain Substance 405, such as drug for example, in the form of liquid, powder or solid to be controlled delivered to the corresponding point on the Target surface. It is further appreciated that pocket 404 may be used to collect and accommodate Substance 405 extracted from the Target surface for further processing, such as biological monitoring or analysis.

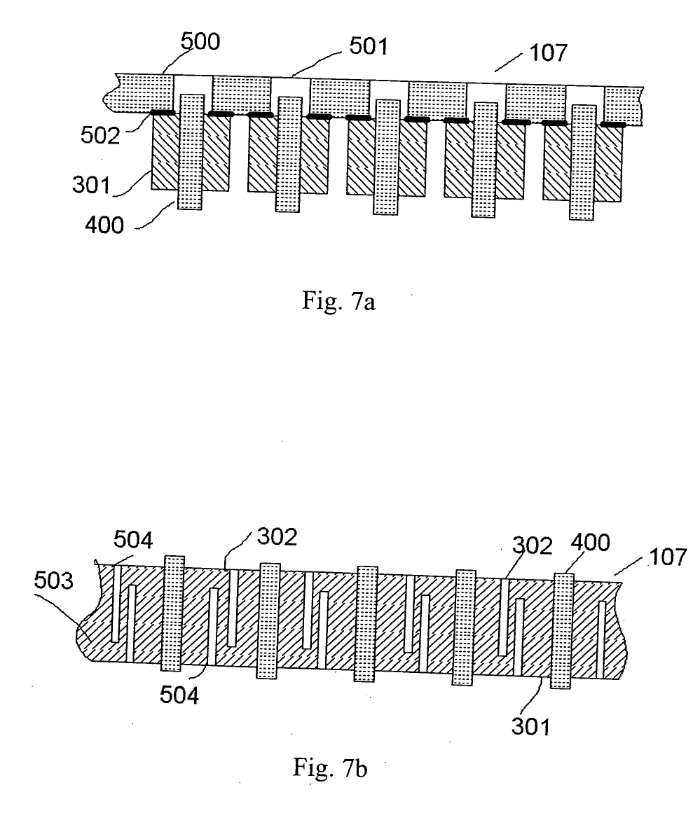

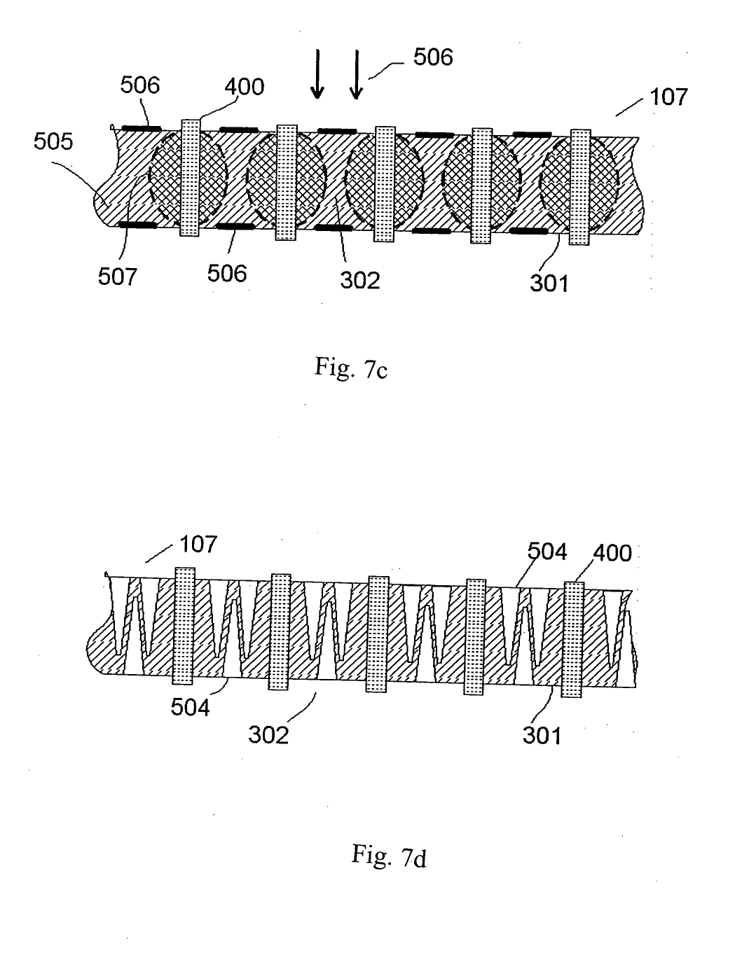

FIG. 7 illustrates some Sheet structure 107 non-limiting examples. The films are preferably made of flexible materials formed of at least one layer, however, multi-layer structures are also beneficial in some applications. The materials may be selected from elastomeric materials including, for example, elastomers such as natural or synthetic rubber, styrene block copolymers containing isoprene, butadiene, or ethylene (butylene) blocks, metallocene-catalyzed polyolefins, polyurethanes or polydiorganosiloxanes. Other elastomers can be related to the following groups: polyesters, polyamides, polyolefins, block and star polymers.

FIG. 7A shows a construction based on a relative thin flexible material foil, such as Latex, rubber or silicon-rubber 500, with openings 501 aligned in registration to Contact probe active Element Array. The More rigid constituents 301 are adhered to the foil 500 with registration to the openings 501 by common adhesion means 502, known to any professional in the field such as contact adhesive materials, welding or fusion. If polymers are selected, some are marketed that contain small amounts of grafted reactive moieties. When properly matched, these materials effectively increase interlayer adhesion. Useful pairs include, for example, carboxylic acid/amine, maleic anhydride/amine, carboxylic acid and maleic anhydride/hydroxyl, maleate and maleic anhydride/double bond, carbodiimide/carboxylic acid, isocyanate/hydroxyl, amine/hydroxyl halide, ester/amine, ester/ester, ester/hydroxyl phenol, amide/ester, epoxide/hydroxyl or amine or carboxylic acid or maleic anhydride, oxazoline/carboxylic acid or phenol or maleic anhydride and lactam/amine or acid ionomer. FIG. 7B shows a construction based on a thicker film 503 wherein controlled depth fractures 504 are made surrounding each More rigid constituent 301. The fracture depths as well as the number of fractures between adjacent More rigid constituents provide the required degree of flexibility.

FIG. 7C shows a construction based on a thick film 505 made of a polymer material on which at least one mask 506 opaque to UV is deposited or printed at least at one side, to avoid UV radiation 506 from curing the masked zones, thus forming polymerized rigid zones 507 functioning as the said More rigid constituent 301, supported by uncured zones 302 which are softer and act as deformable zones allowing relative displacement upon exerting force.

FIG. 7D shows another cross section structure in which the foil thickness around the More rigid constituent is reduced 504 to provide the require flexibility. Such a construction may be made in various techniques known to professionals in the field of material processing, such as molding, masked etching, selective dissolving, heat printing and stamping techniques.

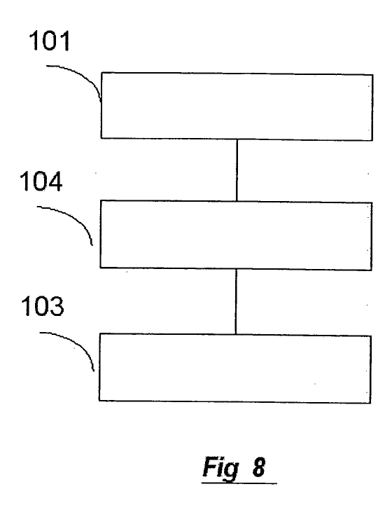

It is appreciated that the present invention constitutes also a method for improving the utilization of contact probs. Contact probes, which by definition involve physical contact with the Target surface, resulting in a possible deterioration of the probe or the Target surface due to a mutual contamination or wear. For example a bed of nails test fixture used in electronics industry is a complex and expensive instrument, being susceptible to wear and contact contamination, adversely affecting its functionality. Another example is an auscultation probe for receiving acoustic signals by attaching it to the skin of a patient. A further other example may be a treatment probe for thermal ablation of the living skin. Reference is made to FIG. 8 wherein a block illustration of a method for improving Contact probe 101 procedures is illustrated. According to the invented method a Contact probe procedure comprising the steps of:
a. selecting a mediating Adaptor structure 104;
b. mounting said mediating Adaptor structure 104 in alignment with the Contact probe 101;
c. performing the intended procedure on the Target surface 103; and then
d. removing the said mediating Adaptor structure 104.

It is appreciated that the mediating Adaptor structure may be selected from a variety of Adaptor structures, 104 illustrated as multiple, specially designed for different functionalities as described above. For example in case of Substance delivery each of the mediating Adaptor structures may hold different Substance and be selected in accordance with the specific circumstances. It is further appreciated that the removed mediating Adaptor structure may be disposed or reprocessed, such as sterilized or refilled for additional use. In case of a disposable type, means for preventing remounting after removal may be utilized (not shown).

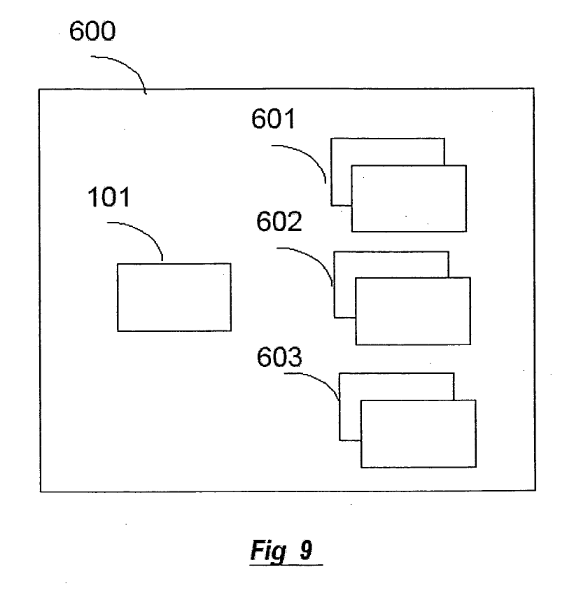

It is further appreciated that the present invention also constitutes a Kit. Reference is made to FIG. 9 wherein a system in the form of a kit is illustrated. In view of the benefits of the Contact probe Adaptor structure and taking into consideration the required mechanical compatibility of the Adaptor structure and the Contact probe it would be desirable to provide a kit 600 comprising at least one Contact probe 101 and at least one compatible adaptor 601. It is preferable in some applications to introduce a kit comprising multiple Adaptor structures 601 designed for disposable use. It is also preferable in some applications to introduce a kit comprising Adaptor structures specially designed for various functionalities 602 and 603. For example the Adaptor structure 602 may reduce the spatial density or provide a special pattern composed of a subset of a given Contact probe Elements according to the specific use. For another example any of the Adaptor structures 603 may accommodate different Substance for delivery. For yet another example the various Adaptor structures may be designed to modify the contact force in accordance to the specific application. It is appreciated that the kit approach provides numerous functional combinations within the scope of the present invention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

It will be appreciated by those skilled in the art that other embodiments of this invention are possible which will not depart from the spirit of the invention as disclosed herein. Accordingly, the invention shall be limited in scope only by the attached claims.

1. An Adaptor structure mountable in alignment to at least two Contact probe front-end Elements, providing a physical buffer between said front-end Elements and a Target surface while substantially transferring the contact force exerted by each of said front-end Elements to the corresponding points on the said Target surface.

2. An Adaptor structure according to claim 1, wherein the said Contact-probe is a part of any combination of systems selected from the list: drug delivery system, ultrasound system, bioelectric system, bioimpedance system, transdermal drug delivery system, phototherapy system, thermo neurology system, auscultation system, electronic testers, vibrator system, heater system and temperature testing system.

3. An Adaptor structure according to claim 1, wherein the said Contact probe front-end Element is a functional element that requires physical contact with a Target surface, with a contact force greater than a predetermined value, for proper operation.

4. An Adaptor structure according to claim 1, wherein the said Contact probe front-end Element is selected as any combination from the group: electrodes, piezoelectric elements, heating elements, cooling elements, vibrating elements, microphonic elements, light emitting elements, light sensing elements, temperature sensing elements, load sensing elements, force actuating elements, Substance delivery elements and Substance receiving elements.

5. An Adaptor structure according to claim 1, wherein the said Target surface is selected from the group: surface of a solid object, surface of a flexible object, printed circuit board, micro electronics die, micro electronics wafer, painted surface, coated surface, biological skin and biological tissue.

6. An Adaptor structure according to claim 1, wherein the complexity of said Adaptor structure is significantly lower than that of the said Contact probe front-end thus suitable for disposable use.

7. An Adaptor structure according to claim 1, wherein the said Adaptor structure is adapted for sterilization.

8. An Adaptor structure according to claim 1, wherein the said Adaptor structure is replaceable.

9. An Adaptor structure according to claim 1, wherein the said Adaptor structure is adapted for one time use only.

10. An Adaptor structure according to claim 1, comprising adhesion means for adhering said Adaptor structure to said Target surface.

11. An Adaptor structure according to claim 1, wherein at least two functional paths between the said front-end Elements and the corresponding points on the said Target surface is provided.

12. An Adaptor structure according to claim 11, wherein the said at least two functional paths are selected as any combination from the group: electrical conducting path, heat conducting path, light conducting path, fiber optic path, wave-guide path, Substance transfer path and porous path.

13. An Adaptor structure according to claim 11, wherein the said at least two functional paths are intently adapted to modify the response of said Contact probe front-end Elements by introducing factors selected from the list: electrical resistance, electrical impedance, electromagnetic radiation loss, acoustic conductance, thermal conductance, mechanical stiffness and Substance permeability.

14. An Adaptor structure according to claim 11, wherein the said at least two functional paths are intently adapted to modify the spatial response of said Contact probe front-end Elements by modifying, including substantially blocking, the functional paths in accordance with a spatial pattern.

15. An Adaptor structure, according to claim 1, comprising a deformable sheet structure allowing local deformations, in the substantially normal to the sheet structure orientation, upon exertion of local force while minimizing the influence on the contact force of surrounding points.

16. An Adaptor structure according to claim 15 wherein the said sheet structure consists of plurality of More rigid constituents in alignment with at least two Contact probe front-end Elements.

17. An Adaptor structure according to claim 15, wherein the said sheet structure material joining the said More rigid constituents, provides Elastically coupling means by any combination selected from the list: reduced material thickness, reduced stiffness by internal cavities or pockets, non-through fractures, masked polymerization, and by utilization of different materials for the Elastically coupling and the said More rigid constituents.

18. An Adaptor structure according to claim 16, wherein a More rigid constituent comprise at least one element selected from the list: electrical conducting element, heat conducting element, light conducting element, fiber optic element, wave-guide element, solid element and porous element.

19. An Adaptor structure according to claim 16, wherein a More rigid constituent comprises at least one cavity to form a Reservoir for a Substance.

20. An Adaptor structure according to claim 17 wherein said Elastically coupling material is selected from the group consisting of natural and synthetic rubbers, styrene block copolymers containing isoprene, butadiene, or ethylene(butylene) blocks, metallocene-catalyzed polyolefins, polyurethanes and polydiorganosiloxanes.

21. An Adaptor structure according to claim 16 wherein one or more of said More rigid constituents are coated with an adhesive material.

22. A method for improving Contact probe utilization by providing a mediating replaceable Adaptor structure in alignment with Contact probe front-end Elements, wherein the said Adaptor structure is characterized by enabling the transfer of the force exerted by at least some of said front-end probe Elements towards the corresponding points on the Target surface while maintaining at least some of the functionalities of the said Contact probe.

23. A method for improving Contact probes utilization according to claim 22, wherein the said mediating Adaptor structure is adapted for a disposable use.

24. A method for improving Contact probes utilization according to claim 22, wherein the said mediating Adaptor structure is adapted for sterilization.

25. A method for improving Contact probes utilization according to claim 22, wherein the said mediating Adaptor structure is adapted to modulate at least one feature of, at least one of the said Contact probe front-end Elements, as sensed at the Target surface.

26. A method for improving Contact probes utilization according to claim 25, wherein the said at least one modulated Contact probe front-end Element feature is selected from the list: contact force, electrical conductivity, contact area, thermal conductivity, acoustical conductivity, light attenuation, and Substance permeability.

27. A method for improving Contact probes utilization according to claim 22, wherein the said mediating Adaptor structure is adapted to modify the spatial pattern of said Contact probe front-end Elements features as sensed at the Target surface.

28. A kit comprising at least one Contact probe and at least one Adaptor structure according to claim 1.

29. A kit according to claim 28 consisting of at least two disposable Adaptor structures.

30. A kit according to claim 28, consisting of at least two Adaptor structures with different characteristics.
